(19) United States
(12) Patent Application Publication
(10) Pub. No.: US 2002/0082591 A1
(43) Pub. Date: Jun. 27, 2002

(54) Device for the treatment of glaucoma

(76) Inventor: Eduard Haefliger, Basel (CH)

Correspondence Address: JORDAN AND HAMBURG LLP, 122 EAST 42ND STREET, SUITE 4000, NEW YORK, NY, 10168, US

(21) Appl. No.: 09/997,738

(22) Filed: Nov. 29, 2001

(30) Foreign Application Priority Data
Dec. 14, 2000 (DE) 100 62 478.2-51

(51) Int. Cl. A61B 18/20

(52) U.S. Cl. 606/6

(57) ABSTRACT

A device for the treatment of glaucoma with a laser catheter and a light-conducting fiber arrangement, into the proximal end of which light can be coupled and at the distal end of which a light-emerging surface is provided which, during the treatment, is opposite to a bent stent, which is disposed in Schlemm's canal, the stent having an essentially triangular cross section in such a manner, that the light-emerging surface is essentially congruent with the opposite side surface of the stent in Schlemm's canal.

### BACKGROUND OF THE INVENTION

The invention relates to a device for the treatment of glaucoma with a laser catheter and a light-conducting fiber arrangement, into the proximal end of which light can be coupled and at the distal end of which a light-emerging surface is provided which, during the treatment, is opposite to a bent stent, which is disposed in Schlemm's canal.

For the treatment of glaucoma, that is, for eliminating the excess pressure in the eyeball, there are drug treatments, as well as a series of surgical methods. Among other things, a device of the above-described type has also already been described, with which, by means of UV light, which preferably is generated by an excimer laser and passed by a glass fiber optical light guide into the interior of the eye, the sponge-like trabecula system, through which the aqueous humor flows from the anterior and posterior chambers of the eye, is removed locally, so that the aqueous humor can reach Schlemm's canal more easily, through which it is finally discharged.

To use this device, it is necessary to open the eye locally, in order to pass light with the help of the light-conducting laser catheter into the immediate vicinity of the tissue of the trabecula system, which is to be perforated. In this connection, it is necessary to position the light-emerging surface precisely in front of Schlemm's canal, in order to perforate the trabecula system precisely at this place and to ensure the discharge in Schlemm's canal. However, this positioning of the distal end of the laser catheter is very difficult and, as a result, makes the use of such a glaucoma treatment device, for which it is important to actually find the correct position for introducing the laser catheter, to some extent a matter of luck. In this connection, it is particularly difficult to ensure that the emerging laser light destroys exclusively the tissue of the trabecula system in front of Schlemm's canal and not that lying next to or behind it. However, when the usual cylindrical stent is used, a corresponding positioning and the avoidance of light, deflected laterally by deflection, is very difficult.

### SUMMARY OF THE INVENTION

It is therefore an object of the invention to develop a device of the type named above further, so that damage to the unaffected tissue next to and behind Schlemm's canal is avoided reliably in a simple manner.

Pursuant to the invention this objective is accomplished owing to the fact that the stent has an essentially triangular cross section, in such a manner that the light-emerging surface essentially is congruent with the opposite side surface of the stent in Schlemm's canal.

By means of the inventive arrangement, the tissue of the trabecular system, during the introduction of the laser catheter through the light-emerging surface forming the tip, is compressed against the side surface of the stent, which is disposed parallel to the light-emerging surface, so that the laser light exclusively burns an opening in the tissue in front of Schlemm's canal without any risk that light reflected laterally can cause damage to the tissue at other places. Starting out from the position of Schlemm's canal, and assuming that the laser catheter is guided essentially frontally to the front surface of the eye, the above-described, special parallel positioning arises owing to the fact that, starting out from an inclination of the light-emerging surface at an angle of 65° with respect to the axis of the light-conducting fiber arrangement, the inner surface, lying on the inside of the arc opposite to the light-emerging surface of the laser catheter, forms an angle of about 115° with the narrow base surface, and the outer surface, lying on the outside of the arc, forms an angle of about 40° with the base surface of the stent.

In a further development of the invention, the stent, which preferably has a bending radius of about 7 mm, corresponding to the bending radius of Schlemm's canal and, matching the diameter of Schlemm's canal, should have a cross-sectional dimension of about 0.15 mm, may be provided at one end with a platelet or a cam, so that it can be gasped by tweezers, pliers, or the like.

Further advantages, distinguishing features and details of the invention arise out of the following description of an example and from the accompanying drawings.

### IN THE DRAWINGS

FIG. 1 shows an enlarged perspective view of an inventive stent,

FIG. 2 shows a diagrammatic partial section through the anterior and posterior chamber of the eye with the inserted stent, the front end of a laser catheter for the treatment of glaucoma being indicated,

FIG. 3 shows an enlarged partial section from FIG. 2 with the stent and the light-conducting fiber arrangement of the laser catheter, which is positioned in front of the stent, and

FIG. 4 shows a partial section, essentially corresponding to FIG. 3, with a modified fiber arrangement, including an adjusting recess,

### DESCRIPTION OF THE PREFERRED EMBODIMENTS

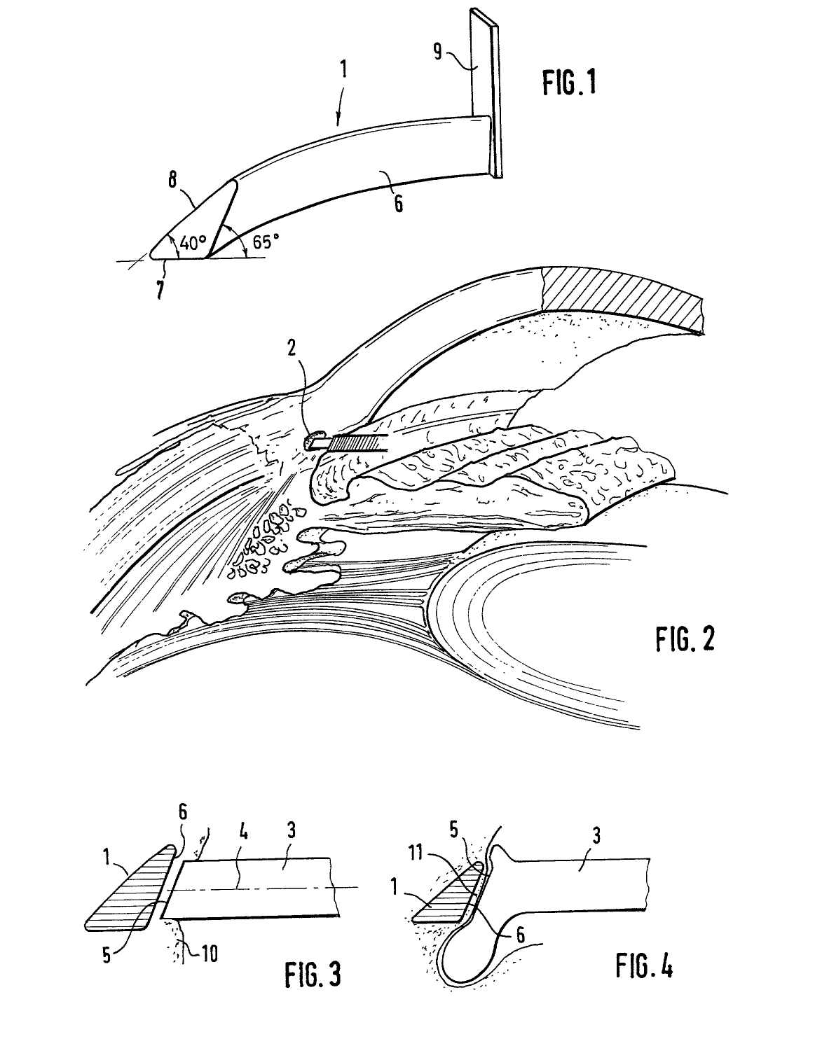

In contrast to previous arrangements, in which the cross section is round, the stent, which is indicated in FIG. 1 and consists, for example, of stainless steel or the like, is essentially triangular in cross section, the front surface 6 of the stent, which, during the positioning in Schlemm's canal 2, lies opposite the end of the light-conducting fiber arrangement 3 of a laser catheter with the light-emerging surface 5 cut at an angle of 65° to the longitudinal axis 4, is inclined at an angle of 115° to the narrow base surface 7, while the averted back surface 8 forms an angle of about 40° with the base surface. The height of the back surface 8 is about 1.8 mm and the total length of the stent is about 7 mm. The bending radius is selected to correspond to the curvature of Schlemm's canal and is about 7 mm. At one end, the stent 1 is provided with a platelet 9, so that it can be grasped by means of tweezers, pliers or the like.

FIG. 3 shows how, by the special parallel orientation of the light-emerging surface 5 to the inner surface of the stent 1 with compression of the tissue 10 of the trabecula system in front of Schlemm's canal, a positioning is achieved, for which laterally reflected light or light of the laser catheter, striking the back side of the Schlemm's canal, can be prevented reliably, so that an opening, through which the aqueous humor can reach Schlemm's canal, through which it finally is discharged, is produced exclusively in front of Schlemm's canal with burning away of the tissue.

FIG. 4 shows an enlarged partial section from FIG. 2, which corresponds essentially to FIG. 3, with a modified fiber arrangement 3, containing an adjusting recess 11. The light-emerging surface 5, formed in an appropriate manner, as in FIG. 3, congruent with the opposite side surface 6 of the stent in Schlemm's canal, that is, disposed parallel to this side surface 6, forms the bottom of the adjusting recess 11 here, which embraces the stent at the top and at the bottom.

What I claim is:

1. A device for the treatment of glaucoma with a laser catheter and a light-conducting fiber arrangement, into the proximal end of which light can be coupled and at the distal end of which a light-emerging surface is provided which, during the treatment, is opposite to a bent stent, which is disposed in Schlemm's canal, wherein the stent has an essentially triangular cross section in such a manner, that the light-emerging surface is essentially congruent with the opposite side surface of the stent in Schlemm's canal.

2. The device of claim 1, wherein the light-emerging surface forms the bottom of an adjusting recess of the fiber arrangement, which embraces the stent at the top and at the bottom.

3. The device of claim 1, wherein the inner side surface, lying on the inside of the arc opposite to the light-emerging surface of the laser catheter, forms an angle of about 115° with the narrow base surface and the outer surface, lying on the outside of the arc, forms an angle of about 40° with the base surface of the stent.

4. The device of claim 1, wherein the stent has a bending radius of about 7 mm.
